(19) United States
(12) Patent Application Publication
(10) Pub. No.: US 2010/0003715 A1
(43) Pub. Date: Jan. 7, 2010

(54) Biological agent detection and identification system

(76) Inventor: Francesco Pellegrino, Cold Spring Harbor, NY (US)

Correspondence Address: WALLACE G. WALTER, 5726 CLARENCE AVE, ALEXANDRIA, VA, 22311-1008, US

(21) Appl. No.: 11/017,042

(22) Filed: Dec. 21, 2004

(51) Int. Cl. C12Q 1/16; C12M 1/34

(52) U.S. Cl. 435/35; 435/288.7

(57) ABSTRACT

A sample under test containing non-live and/or live particulates is subject to optical excitation on a single particle-by-particle basis or as a small group of particulates sufficient to induce a subsequent fluorescence emission that is observed for a selected period of time by a sensor, typically a photomultiplier tube. The output of the sensor is representative of the intensity or amplitude of the fluorescence emission while the decrease in that intensity or amplitude with time is representative of the decay rate of the fluorescence emission. Those particulates exhibiting a decay rate “faster” than a threshold decay rate, which is determined empirically for the class of biological agents of interest, are identified as living while those particulates exhibiting decay rate “slower” than a threshold decay rate, which is also determined empirically for the class of biological agents of interest, are identified as a non-live interferant.

### BACKGROUND OF THE INVENTION

The present invention relates to the detection of biological agents and, more particularly, to the detection of biological warfare agents using the decaying fluorescence signal emitted by such agents after irradiation by a suitable optical energy source.

Various systems are known for detecting the presence of biological agents in particulate form. Biological aerosol warning systems (BAWS) detect the presence of biological agents by measuring the sudden increase in the respirable particle count (usually for particles in the 2-10 micron range) above a background reference level. Other systems, such as Ultra-Violet Laser Induced Fluorescence (UV-LIF) detectors measure the increase in fluorescence emission subsequent to laser excitation of the particles in their biological action spectrum. One issue with UV-LIF detectors is that the presence of non-biological particulates or non-live particles that also provide a fluorescence emission can ‘mask’ as live biologic particles and thus trigger false positive alerts.

### SUMMARY OF THE INVENTION

A method and system for the detection of biological agents, including biological warfare agents, irradiates suspect particulates with optical excitation, for example, from a laser to effect fluorescence emission therefrom while measuring or assaying the fluorescence emission as a function of time. The method and system discriminates between live and non-live particulates by recognizing that the fluorescence emission from live particulates decays substantially faster than that for non-live particulates. By setting some threshold value intermediate to the decay rates for live and non-live particulates, a fluorescence emission that decays above that threshold value can be discarded as representative of a non-live particulate while a fluorescence emission that decays below that threshold value can be considered as representative of a live biologic particulate of interest thereby reducing false positive alerts.

In an illustrative embodiment, a sample under test is directed through a small cross-section lumen or test chamber such that any particulates will move as a single particulate or as a small group of spatially separated particulates therealong. A source of optical excitation energy irradiates the particles to induce a subsequent fluorescence emission that is observed for a selected period of time by a sensor, typically a photomultiplier tube. The output of the sensor is representative of the intensity or amplitude of the fluorescence emission while the decrease in that intensity or amplitude with time is representative of the decay rate of the fluorescence emission. For example, fluorescence emission of a live biologic particulate may undergo decrease in intensity to 1/e after 200 picoseconds while the corresponding fluorescence emission for a non-living biologic or non-biologic particulate may undergo a change of more than 1/e of its initial value over a longer time period. Thus, by evaluating the decline in intensity over some time period, a quantitative “figure of merit” can be obtained to discriminate between live and non-live particles. Optionally, other techniques can be used, including, for example, Fourier transform analysis to identify the waveform of the fluorescence emission decay or any waveform coefficients to discriminate between live and non-live particulates in the sample under test.

Once a determination has been made that a sample under test includes a live particle or particulates, the sample under test can be selectively directed to a suitable substrate, such as a collagen-coated microscope slide, where the suspect particles are accumulated. An optical system that includes an image acquisition or capture device, such as an analog or digital still or moving image camera, obtains an image of the accumulated particle or particles and subjects that image to pattern-recognition software, such as an appropriately trained neural network, to conduct a preliminary classification of the particle or particulates. Additionally, the so-acquired image can be directly viewed or transmitted to a remote location for viewing and assessment by a trained observer.

### BRIEF DESCRIPTION OF THE DRAWING

FIG. 1 is an overall schematic view of a preferred or illustrative embodiment of the detection system;

FIG. 1a is an example of an alternate organization for the principal components of the system of FIG. 1;

FIG. 1b is an example of another alternate organization for the principal components of the system of FIG. 1;

FIG. 2 is a detail schematic view of the image viewing arrangement of FIG. 1;

FIG. 3 is an overall schematic view of a distributed network arrangement;

FIG. 4 is a graphical representation of the difference in the decay rate of a fluorescence signal emitted by live and non-live particles (in solid-line) with the horizontal axis representing time in picoseconds (ps) and the vertical axis representing signal intensity in arbitrary units and also showing several intermediate threshold values (in dotted-line); and

FIG. 5 is a simplified flow diagram showing one way in which the system of FIG. 1 can be controlled.

### DESCRIPTION OF THE PREFERRED EMBODIMENT

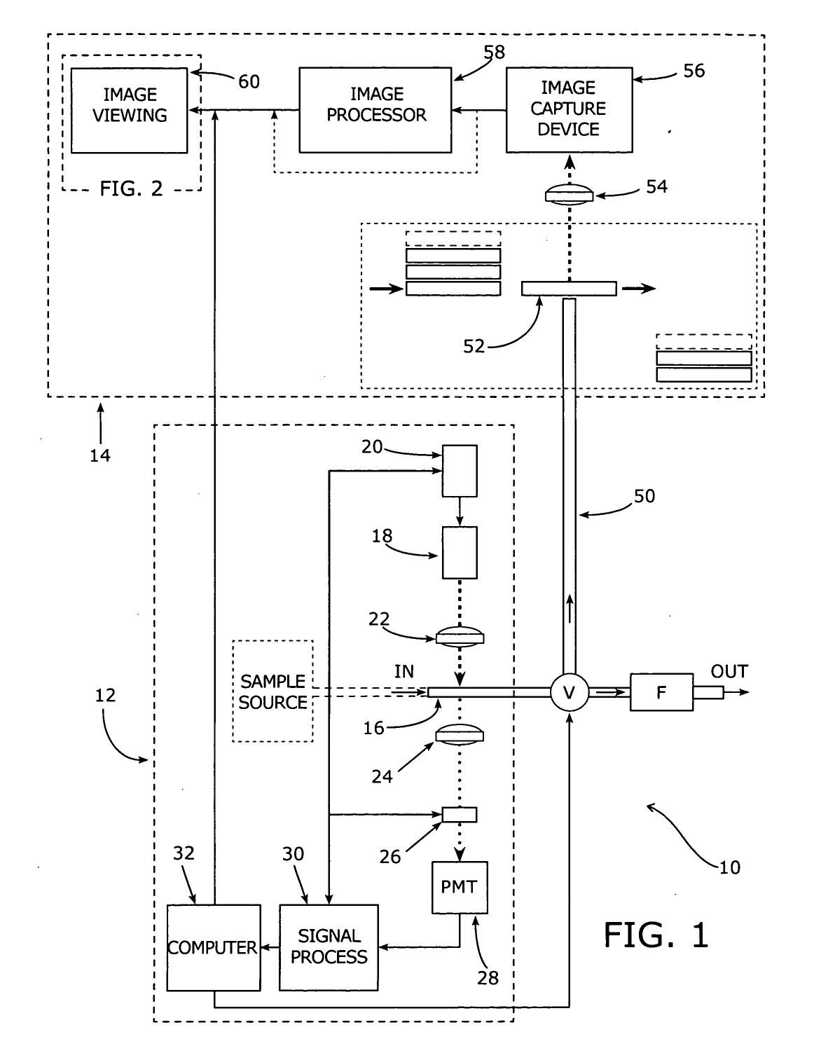

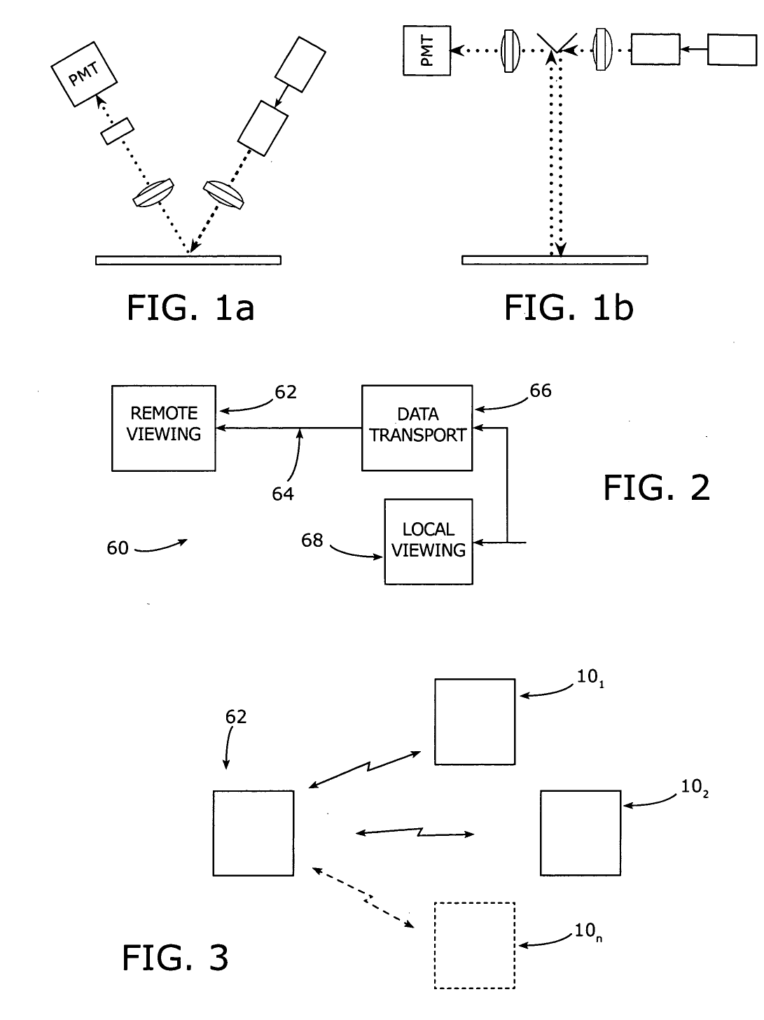

The detection system of the present invention is shown in schematic form in FIG. 1 and designated generally therein by the reference character 10. As shown, the system 10 includes a fluorescence analysis subsection 12 designed to assay or interrogate a sample gas flow for the presence of particulates, including bioagent particulates, and an imaging section 14 designed to acquire or capture images of any particulates deemed to be of interest, including particles of a biological nature as detected by the fluorescence analysis subsection 12.

The fluorescence analysis subsection 12 includes a testing volume or chamber in the form of a hollow tube 16, fabricated, for example, from glass or quartz or Teflon-coated vinyl tubing having an internal bore sufficiently small that particulates in the sample under test travel through the bore or lumen thereof at a concentration preferably allowing the particles to be spatially separated from one another so that an excitation pulse can excite each particle individually and measure its corresponding fluorescence emission or signal on a particle-by-particle basis. In general, a bore diameter on the order of a mm. is suitable; while a larger bore tube 16 is also suitable, it is believed that system resolution may decline with increased bore diameter. The tube 16 connects to a valve V, the function of which is discussed in more detail below, to an exhaust filter F and to an exhaust OUT. If desired, the exhaust filter F can be of the type that captures all particles to assure that any particulate bioagent or fragment thereof will not escape the system. As an option, the filter F can include chemical biocides or other means to kill or otherwise render inactive any bioagent in the gas flow. As shown in dotted-line illustration, the system 10 includes a known gas-handling system or arrangement “SAMPLE SOURCE” for introducing a measured or metered flow of the sample under test into the input end IN of the tube 16; these known gas-handling devices can include a pump, pressure and/or flow regulator(s), related ON/OFF and proportional metering valves, and a controller to provide a continuous or intermittent controlled flow-rate gas-flow into the tube 16. Additionally, the gas-handling system can include a source of particulate-free gas (preferably an inert or non-reactive gas) that can be used to periodically purge the system or to separate test sample flows in those cases where the samples under test are transported through the tube 16 on a non-continuous or intermittent basis. Additionally, the gas-handling system can include a source of calibration gases having known concentration(s) of fluorescent and non-fluorescent agents in order to calibrate the apparatus.

The sample under test is excited by radiation that, as explained below, serves to excite any particulates in the tube 16 to yield a fluorescence emission, signal, or ‘signature’ that may be measured by a suitable optical sensor. More particularly, an excitation source 18, such as a laser, is controlled by a trigger unit 20 to emit one or more controlled-duration pulses of excitation energy (dashed line in FIG. 1) through a suitable optical system, symbolically represented at 22, into the tube 16. The optical system 22 can include one or more lenses, mirrors, and/or filters, etc. to pre-condition the excitation energy prior to entry into the tube 16, if and as desired. While the embodiment of FIG. 1 includes a selectively triggered laser and as can be appreciated, an alternate arrangement can include a continuous laser in which a selectively controlled electro-mechanical shutter, acousto-optcial gate, or electro-optical gate interrupts the laser beam in a controlled manner.

As explained in more detail below, the excitation energy serves to excite any particulates within the tube 16, including both inorganics and live and not-live organics. As a consequence of this excitation irradiation, many of those particulates will provide a fluorescence emission (dotted line in FIG. 1). The fluorescence radiation is passed though an optical system 24, symbolically represented as a lens, that includes one or more lenses, mirrors, and/or filters, etc. to precondition the fluorescence radiation, if and as desired. A controllable electro-mechanical shutter or electro-optical gate 26 is provided to interrupt the fluorescence radiation, as discussed below, entering into a radiation sensor 28, such as a conventional photomultiplier tube (PMT). The output of the sensor 28 is provided to a fluorescence signal processor 30 that, as explained below, provides initial processing of the sensor 28 output for use by a control computer 32. The fluorescence signal processor 30 and the computer 32 can take the form of software- and/or firmware-controlled microprocessor(s), an appropriately configured ASIC (application specific integrated circuit), discrete logic, or a combination thereof. In addition, data can be stored in and/or retrieved from various memory devices including traditional hard disc storage, various types of static RAM (SRAM), or dynamic RAM (DRAM). While the preferred embodiment utilizes a separate fluorescence signal processor 30 and computer 32, as can be appreciated by those skilled in the art, the functions executed by these devices can be combined in a single processing device or, if desired, distributed among a group of interconnected or interoperated processing devices.

In the arrangement described above and shown in FIG. 1, the entry into the sensor 28 of any fluorescence signal emitted by the particles in the tube 16 is controlled by a shutter or gate 26; as can be appreciated there may be arrangements in which the sensor is ‘ungated’ so that all radiation is allowed to enter the sensor with signal processor 30 and/or computer 32 functioning to discriminate between the fluorescence signal and the output of the laser 18. Additionally, control of the irradiating optical energy and the resulting fluorescence signal can be accomplished by one or more out-of-phase rotating slotted or apertured discs that ‘chop’ the irradiating optical energy while blocking the input to the sensor 28 and, conversely, block the irradiating optical energy while unblocking the input to the sensor 28. The organization of FIG. 1 can be viewed as an “inline” system. Other arrangements are equally suitable, including, for example, the non-inline organizations of FIG. 1a and FIG. 1b; in such other arrangements, the optical systems are reconfigured as appropriate.

The imaging subsection 14 is designed to capture images of any particulates in the sample under test, and, in particular, those particulates that are deemed to be bioagents or possible bioagents of interest. The image subsection 14 includes a gas flow transport tube 50 that is connected between the valve V and an appropriately housed collection slide 52. The valve V is selectively actuatable by an appropriate control signal so that the sample under test can be directed to the tube 50 or to the filter F and the connected exhaust OUT. The collection slide 52 preferably has a coating or deposit of a suitable material, i.e., collagen, to capture and hold any particulates in the sample under test; a nutrient component or a biocide or staining agent(s) are not excluded as additional components that can be associated with the collection slide 52.

The collection slide 52 may be part of a larger slide transport system by which a series of individual slides can be presented to collect particulates. For example and as shown in schematic form in FIG. 1, a magazine-type or stack-like supply of fresh slides 52 can be positioned adjacent to one side of the tube 50 (i.e., on the left in FIG. 1) and pushed into position by a suitably controlled actuator (not shown) to collect particulates while the immediately proceeding slide is pushed into or moved into a receiving container or station.

The image capture system 14 is located relative to the collection slide 52 to capture an image of any collected particulates. As shown, the image capture system includes a lens arrangement 54, such as a microscope-type objective, and an image capture device 56 in the form a color CCD-type still or moving digital camera or similar imaging device. The lens arrangement 54 can include lens options of different magnifications, depths of field, filters of various types including polarizing filters, and, if additionally desired, different wavelength illumination to capture different optical characteristics of the captured particles. While a digital type still camera is preferred, any type of analog or digital still or moving image camera of sufficient resolution is acceptable. While not shown in FIG. 1, a photographic film camera can be provided as an adjunct to the image capture device 56 to provide a conventional film record of any collected particulates.

The output of the image capture device 56 is provided to an image processor 58 that, in the preferred form, is a software or firmware-based processor that functions to enhance the image by performing one or more of a multitude of commercially available image processing techniques, such as thresholding, edge enhancement, or other image filtering processes in order to assist in classifying the collected particulates by distinguishing their optical and physical characteristics. Preferably, the image processing includes a pre-trained or trainable neural network or wavelet classifier that assesses the image field until a probable identification is made. Neural networks are conventionally trained by providing repeated trial exposures to “exemplars” having the optical and/or properties of the particulates of interest until such time that the neural network can recognize and discriminate between the exemplars of interest. While neural network and/or wavelet processing is preferred, other classification systems are not excluded.

The output of the image processor 58 is provided to an image viewing system 60, the details of which are shown in FIG. 2. As shown in FIG. 2, the output of the image capture device 56 (as optionally processed by the image processor 58) can be provided to a remote viewing station 62 through a communications link 64 and/or to a local viewing station 68, which is typically co-located or in operational proximity to the fluorescence analysis subsection 12. In each case, the viewing station includes a monitor or other display screen of sufficient resolution and, preferably, color acuity, to allow visual identification of the suspect particulates by a sufficiently trained observer.

As shown by the dotted-line extending from the input to the output of the image processor 58 in FIG. 1, the “raw” unprocessed image information can also be provided to the image viewing system 60.

In the preferred embodiment of the invention, the image information is prepared for transport over the communications link 64 by data transport processor 66 and transported over the communications link 64 to a receiver (not shown) at the remote viewing station 62. The communications link 64 can take any suitable form provided the image data can be transported with sufficient fidelity to the destination. For example, communication can be effected by dedicated wire or optical lines, Internet and/or intranet links, the PSTN, radio-frequency links, or a combination thereof. The remote viewing arrangement discussed above allows for the use of multiple systems 10 in a network-type arrangement. For example and as shown in FIG. 3, a plurality of systems, 10₁, 10₂, . . . 10ₙ, can be located at geographically dispersed locations (including a nationwide or trans-national distribution) and can report via their communications links to a central location that includes a remote viewing station for each system 10₁, 10₂, 10ₙ₋₁, . . . 10ₙ, or, more preferably, a remote viewing system that can be used to display the image information from one or more systems 10₁, 10₂, 10ₙ₋₁, . . . 10ₙ, on an as needed basis.

The present invention recognizes the difference in temporal fluorescence between inorganic or non-living organic particulates and living microorganisms. In particular, it utilizes the measurement of fluorescence lifetime to distinguish between living and non-living microorganisms. Currently available Ultra-Violet Laser Induced Fluorescence (UV-LIF) detectors utilize the measurement of fluorescence emitted from particles excited by Ultra-Violet laser excitation pulses to distinguish between particles containing UV absorbing compounds such as NADH, flavins DNA, and or tryptophan, among others, and inorganic particles which do not fluoresce when excited by such ultra-violet radiation. However, it is well known that certain inorganic materials also absorb such radiation and fluoresce in similar ranges as biological agents, thus acting as interferants. It is an object of the present invention to distinguish between these interferants and living biological agents thereby increasing the signal to noise ratio and improving the sensitivity of UV-LIF detectors to detect biological agents and further to identify those agents as living organisms.

The ability to discriminate living from non-living matter is based on the following principles: When a photon from an excitation source, such as a laser, excites a molecule or microorganism containing a fluorophore in the ground state, the fluorophore can absorb the photon and jump to a higher vibrational energy level of the electronically excited singlet state. This transition from the ground state to the higher excited state takes place on the order of about 10⁻¹⁵ second and is dependent on whether the exciting photon energy matches the energy difference between the ground state and the excited state. Subsequent to excitation, the molecule may undergo relaxation by transferring the energy to lower vibrational energy levels of the excited state, which can occur on the order of a few picoseconds per transfer. Alternatively the energy may be transferred to a triplet state in the same molecule (which later decays as phosphorescence on the order of seconds), or to a lower energy level of a neighboring molecule, or back to the ground state. The emission of light from the excited state is called fluorescence, and the time it takes for the fluorescence to decrease to 1/e of its original value is called the fluorescence lifetime, which is a measure of the time the energy stays in the excited state before returning to the ground state. The fluorescence lifetime ranges on the order of hundreds of picoseconds to hundreds of nanoseconds depending on the energy transfer processes available for de-excitation. The decay of the fluorescence intensity F may thus be represented as

F=Fₒe^{(-t/τ)}

where F and Fₒ are the intensities at time t and at time t=0, and where τ is the excited state fluorescence lifetime.

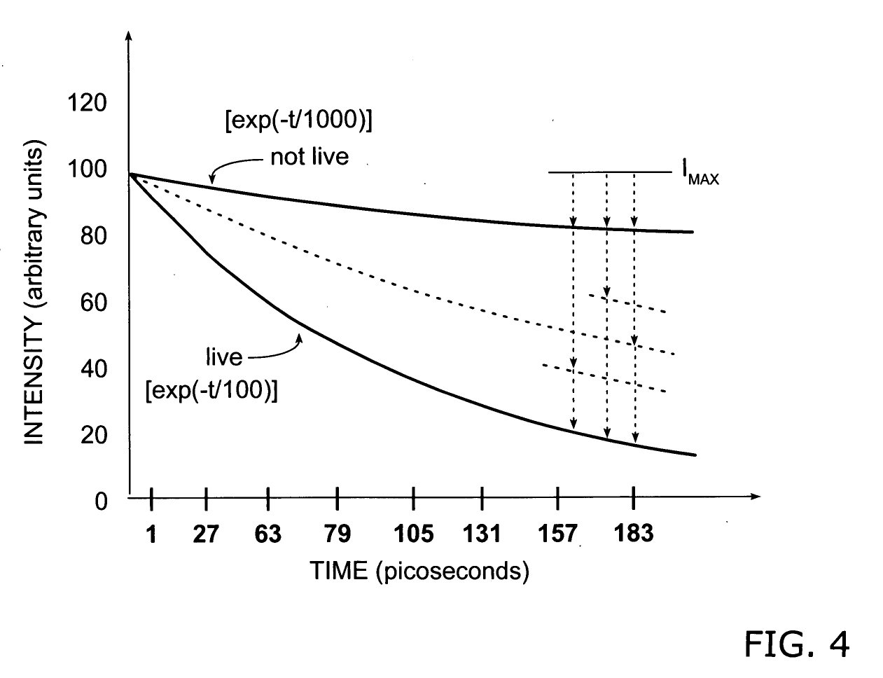

If a molecule has many ways in which to transfer its energy out of the excited state (as in the case of a live organism which has active neighboring molecules or many active vibrational or rotational levels), the rate of energy transfer out of the excited state is high. This is due to the fact that the rate is proportional to the number of possible decay pathways. Correspondingly, the fluorescence lifetime for that molecule, which is inversely proportional to the decay rate, is low. This is shown in FIG. 4 in which the vertical axis represents the intensity of the fluorescence signal while the horizontal axis represents elapsed time. As shown, relatively faster decaying exponential curve [exp(−t/100)] with a lifetime of 100 ps (0.1 nanosecond) is representative of the fluorescence lifetime measured from a living microorganism while the relatively slower decaying exponential curve [exp(−t/1000)] is representative of a non-living particle.

Conversely, if a molecule has few ways in which to transfer its energy out of the excited state (as in the case of a non-living microorganism which has few active neighboring molecules, or few active vibrational or rotational modes because of its decayed state) the rate of energy transfer out of the excited state is low. For this latter case, the fluorescence lifetime is therefore long. This is shown in FIG. 4 by the longer decaying exponential with a lifetime of 1000 ps (1 nanosecond) representative of the fluorescence lifetime measured from a non-living microorganism. In general, a determination of a fluorescence lifetime may be obtained for each particular organism or class or classes of organisms to enable a threshold lifetime to be determined sufficient to discriminate between live and non-live particles.

For any class of organisms, there is some intermediate curve (as symbolically represented by the dashed line in FIG. 4) that can also function as a threshold or threshold indicia for making a live/not-live determination. As shown by the vertical dashed-line arrows on the right in FIG. 4, the quantitative decrease in intensity with time from the initial maximum value at T=0 can be used to make the live/not-live determination. Additionally, the live/not-live determination can be made by determining the live/not-live waveform or waveshape (by FFT) and comparing the measured value with a threshold intermediate waveform or waveshape (for example, as represented by the dashed-line curve in FIG. 4).

The lifetime measurements are obtained by utilizing a photomultiplier tube (sensor 28 in FIG. 1) with picosecond temporal resolution capability (such as available from the Electron Tube Division of the Hamamatsu Corp., Hamamatsu City, Japan and Bridgewater, N.J. 08807). Additionally the signal processing element 30 discriminates whether the lifetime measured for each particle passing through the fluorescence analysis subsection 12 is either greater than or less than a set threshold which may depend on such factors as size, shape, wavelength or other criteria which may be established for setting the living/non-living threshold for a given microorganism. The computer 32 subsequently establishes an alert or an alarm based on the detection of particle or particles satisfying the temporal fluorescence threshold criteria.

With respect to FIG. 1, in normal operation, the valve V is set to allow gas flow to the filter F and to the exhaust side of the system. Individual samples under test can be processed periodically on a recurring or a non-recurring basis according to a timing schedule; a source of particle-free purge gas can be used to separate samples under test or to calibrate the apparatus. Optionally, a gas flow can be continuously passed into the input end IN of the tube 16 (FIG. 1) to pass beneath the optical system 22 as a continuing flow. The triggering unit 20 is operated to turn the optical energy source 18 ON for a selected period of time such that the source excitation pulse is smaller than the fluorescence lifetime; the transport velocity of the particulates in the tube 16 being sufficiently low so that the same particles within the field of view of the optical system 22 will experience the full radiation from the optical energy source 18 during its ON period. With respect to the direct in-line signal detection part of the fluorescence analysis subsection 12 of FIG. 1, it is understood that a more convenient and efficient signal acquisition scheme would utilize signal detection perpendicular to the line of excitation, in order to obviate the excitation signal from entering the detector 28 (as illustrated in symbolic form in FIGS. 1a and 1b), or additionally utilize spectral filtration to isolate the background and excitation source from the emitted fluorescence, or utilize a continuous wave laser or any of a multitude of time resolved fluorescence spectroscopy measurement techniques that may be obvious to those skilled in the art. While the direct in-line measurement technique is described herein and considered a preferred technique, other measurement techniques are equally suitable.

For the configuration illustrated, during the time that the optical energy source 18 is ON, the optical gate 26 is closed. In general, the optical radiation can be anywhere in the optical spectrum between ultraviolet and infrared and must have an energy density high enough to cause the fluorescence effect. Of course, the energy density should not be high enough to damage or pyrolyze any particulates. In general, a laser with an output between 250 and 450 nanometers and a power level of 10 mW is adequate depending on the particular absorption band desired to be excited in the fluorophore of the organism of interest.

Once irradiation is accomplished, the optical energy source 18 is turned OFF while the optical gate 26 is opened immediately or shortly after the optical energy source 18 is turned OFF. Any fluorescence radiated from the now-energized particulates pass through the optical system 24 and enter into the radiation sensor 28. The transport velocity of the sample under test should be sufficiently slow so that the fluorescent particles are in the field of view of the optical system 24 and the radiation sensor 28 until the fluorescence of slowest decaying particle is substantially completed or at least sufficiently completed to insure discrimination between the fluorescence emission decay of live and non-live particles; this period of time generally being determined empirically. The signal processor 30 analyzes the signal output of the optical sensor 28 on a temporal basis to determine whether the decay rate, in the case of the illustrated embodiment, is less than a threshold value X as may be established empirically, indicating the presence of live bioagent or greater than the threshold value X, or other threshold as may be established empirically, indicating a non-living particulate. In that case where a mix of living and non-living particles are in the sample under test, the signal processor 30 can optionally pick-out the faster decaying signal or discriminate between the faster and the slower decaying signals by Fast Fourier Transform and/or by wavelet analysis that computes or otherwise finds the characteristic waveform or shape and discriminate therebetween.

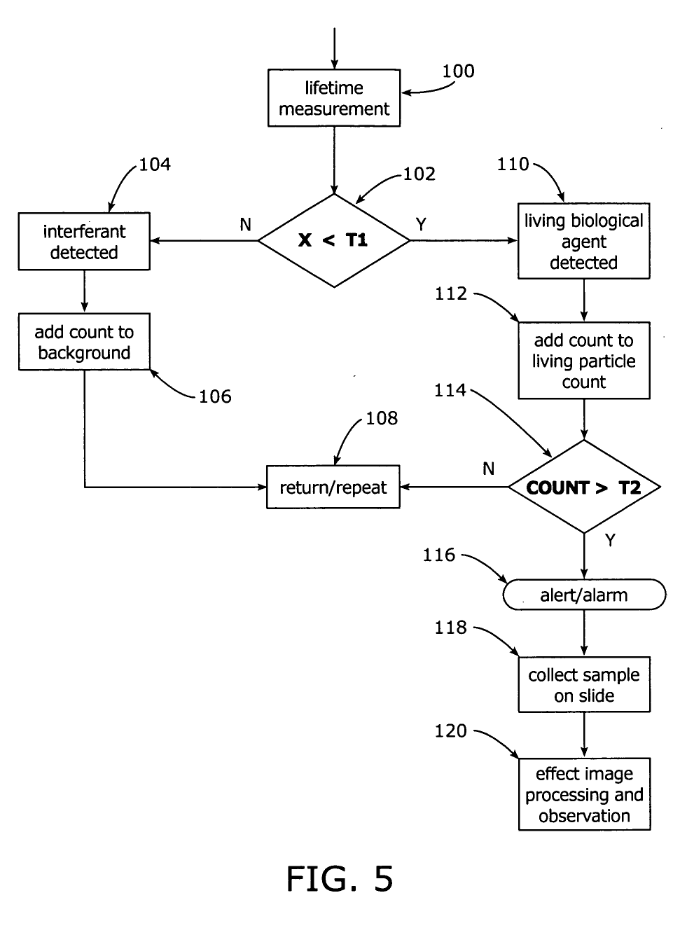

FIG. 5 illustrates a representative process for analysis of the output of the optical sensor 28 to arrive at a live/not-live decision. As shown, the fluorescence lifetime of each successive particle is measured at step 100 and compared against a threshold value T1 (i.e., at about 500 ps in the case of the preferred embodiment) at comparison step 102. In that case where the fluorescence lifetime is greater than the threshold T1 (indicating the detection of an interferant at step 104), that information can be added to an incrementing interferent counter at step 106 and the process repeated at step 108. Conversely, where the lifetime fluorescence is measured at step 100 is less than the threshold T1 (indicating the detection of a probable live particle at step 110), that information can be added to an incrementing “live particle” counter at step 112. The cumulative “live particle” count is compared against some threshold value T2 at step 114. Where the cumulative “live particle” count is less than the threshold value T2, the process is repeated via step 108. Conversely, where the cumulative “live particle” count is greater than the threshold value T2, the process sets an alarm or alert indication at step 116. Thereafter, the computer 32 controls the valve V to redirect the flow of the sample under test into the tube 50 to and toward the collection slide 52 (step 118) with image processing and local or remote observation (or both) effected at step 120.

As mentioned above, the collection slide 52 includes a coating, such as a collagen, that captures or immobilizes particulates delivered to it from the end of the tube 50. As part of the process by which the now-suspect sample under test is transported to the collection slide 52, a particle-free purge gas can also be introduced into the input end IN of the tube 16. After a period of time adequate to ensure that a sufficient quantity of the sample under test has been transported to and accumulated on the collection slide 52, the image capture device 56 captures one or more images of the particulates immobilized in or on the collection slide 52. The magnification or magnification range of any lens in the optical system 54 is typically in the 100× to 500× range. The image processor 58 seeks to provide a preliminary classification, if not a specific identification of the suspect particulates. In the preferred form of the invention, the image processor 58 is an appropriately trained neural network classifier trained to distinguish selected characteristics of various bioagents, such characteristics residing in a bioagent data base providing means whereby a preliminary assessment as to the identity of the bioagent under test may be made, along with an error probability assessment as to the correct likelihood of the classification based on the number of features identified on the bioagent under test relative to the reference feature set and a weighted value of the relative importance of each feature. The image that is captured and any preliminary classification can then be sent to a local monitor or display device or, in accordance with the preferred embodiment, to a remote monitor or display device for viewing by a trained observer for further analysis.

As will be apparent to those skilled in the art, various changes and modifications may be made to the illustrated embodiment of the present invention without departing from the spirit and scope of the invention as determined in the appended claims and their legal equivalent.

1. A method for discriminating between living microorganisms and non-living microorganisms in a gaseous sample having both living microorganisms and non-living microorganisms therein as a function of a fluorescence lifetime less than a selected temporal threshold of about 1000 picoseconds for living microorganisms or greater than said selected temporal threshold for non-living microorganisms, comprising:
a step for flowing the gaseous sample through a lumen of sufficiently small cross-section to substantially spatially separate any living microorganisms and non-living microorganisms in the gaseous sample;
a step of irradiating the gaseous sample within the lumen with optical radiation at least sufficient to induce a fluorescence emission in any living microorganisms and non-living microorganisms in the gaseous sample that have a fluorescence characteristic;
a step for measuring the fluorescence lifetime of any fluorescence emission in response to said irradiating step on a temporal basis; and
a step for comparing the fluorescence lifetime with said temporal threshold to identify those microorganisms having a fluorescence lifetime of less than said temporal threshold and those microorganisms therein having a fluorescence lifetime greater than said temporal threshold.

2. The method of claim 1, wherein said threshold is about 500 picoseconds.

3. The method of claim 1, wherein said threshold is about 100 picoseconds.

4. The method of claim 1, wherein the measuring step further comprises measuring the decrease in an intensity characteristic of the fluorescence emission over a selected period of time.

5. (canceled)

6. (canceled)

7. The method of claim 1, further comprising a step for collecting at least some of any microorganisms in the gaseous sample identified by said comparing act as having a fluorescence lifetime of less than said temporal threshold.

8. The method of claim 7, further comprising a step for acquiring an image of the collected microorganisms and subjecting that acquired image to a stored-program controlled process to classify the collected microorganisms as being of one of a set of microorganisms identifiable by said stored-program controlled process.

9. The method of claim 7, further comprising a step for acquiring an image of the collected microorganisms and conveying that image to a viewing device.

10. A method for discriminating between living microorganisms and non-living microorganisms in a gaseous sample having both living microorganisms and non-living microorganisms therein as a function of a fluorescence lifetime less than a selected temporal threshold of about 1000 picoseconds for living microorganisms or greater than said selected temporal threshold for non-living microorganisms, comprising the acts of:
flowing the gaseous sample through a lumen of sufficiently small cross-section to substantially spatially separate any living microorganisms and non-living microorganisms in the gaseous sample;
irradiating the gaseous sample within the lumen with optical radiation at least sufficient to induce a fluorescence emission in any living microorganisms and non-living microorganisms in the gaseous sample that have a fluorescence characteristic;
measuring the decrease in any fluorescence emission in response to said irradiating act on a temporal basis; and
comparing the decrease in any fluorescence emission with said temporal threshold to identify those microorganisms having a fluorescence lifetime of less than said temporal threshold and those microorganisms therein having a fluorescence lifetime of greater than said temporal threshold.

11. The method of claim 10, wherein said threshold is about 500 picoseconds.

12. The method of claim 10, wherein said threshold is about 100 picoseconds.

13. The method of claim 10, wherein the measuring act further comprises measuring the decrease in an intensity characteristic of the fluorescence emission over a selected period of time.

14. (canceled)

15. (canceled)

16. The method of claim 10, further comprising the act of collecting at least some of any microorganisms in the gaseous sample identified by said comparing act as live particles.

17. The method of claim 16, further comprising the act of acquiring an image of the collected microorganisms and subjecting that acquired image to a stored-program controlled process to classify the collected particles as being of one of a set of microorganisms identifiable by said stored-program controlled process.

18. The method of claim 16, further comprising the act of acquiring an image of the collected microorganisms and conveying that image to a viewing device.

19. A system for detecting the presence of live particles in a gaseous sample, comprising:
a sample chamber into which a gaseous sample is introduced;
an optical radiation source irradiating at least a portion of the gaseous sample in the sample with sufficient irradiation energy to at least induce a fluorescence emission in any live particles in the gaseous sample that have a fluorescence re-radiation characteristic;
a sensor for sensing the intensity of any fluorescence emission from any fluorescing particles in the sample chamber; and
a stored-program controlled processor connected to the sensor for measuring a characteristic associated with the decrease in intensity of the fluorescence emission as a function of time and making a determination as to whether the measured characteristic is consistent with a live fluorescing particle.

20. The system of claim 19, wherein the sample chamber comprises a linearly extending lumen having a sufficiently small cross-section to substantially spatially separate any particles in the gaseous sample.

21. The system of claim 19, wherein the measured characteristic is the decrease in intensity of the fluorescence emission with time and the determination comprises comparing that decrease in intensity of the fluorescence emission at a selected time after irradiation to a threshold indicia indicative of live particles.

22. The system of claim 19, further comprising a substrate having a material thereon for collecting particles in said sample chamber.

23. The system of claim 22, further comprising an imaging device that acquires an image of the particles collecting on the substrate.

24. The system of claim 23, further comprising a stored-programmed controlled processor for analyzing the image of the particles collected on the substrate and classifying the particles collected as being of one of a set of particle types identifiable by said stored-program.

25. The system of claim 23, further comprising an imaging display for displaying the acquired image to a human observer.

26. An apparatus for detecting the presence of live particles in a gaseous sample, comprising:
means for accepting a gaseous sample in a confined volume;
means for irradiating the gaseous sample with optical radiation at least sufficient to induce a fluorescence emission in any live particles in the gaseous sample that have a fluorescence re-radiation characteristic;
means for measuring the decrease with time of any fluorescence emission; and
means for comparing the decrease with time in any fluorescence emission with a threshold indicia that is indicative of the fluorescence emission of a live particle.

27. The apparatus of claim 26, wherein said first-mentioned means flows the gaseous sample through a lumen sufficiently small to substantially spatially separate any particles in the gaseous sample.

28. The apparatus of claim 26, wherein said measuring means measures the decrease in an intensity characteristic of the fluorescence emission at a selected time after irradiation.

30. The apparatus of claim 26, wherein said measuring means measures the decrease in an intensity characteristic of the fluorescence emission over a selected period of time.

31. The apparatus of claim 26, wherein said measuring means measures the decrease in an intensity characteristic of the fluorescence emission over a selected period of time and determining any characteristic waveshape or waveform associated with the decrease in the intensity characteristic of the fluorescence emission over the selected period of time.

32. The apparatus of claim 31, wherein the comparing means compares any said characteristic waveshape or waveform with a threshold waveshape or waveform that is indicative of the difference between a waveshape or waveform indicative of live particles and a waveshape or waveform indicative of non-live particles.

33. The apparatus of claim 26, further comprising means for collecting at least some of any particles in the gaseous sample identified by said comparing act as live particles.

34. The apparatus of claim 33, further comprising means for acquiring an image of the collected particles and subjecting that acquired image to a stored-program controlled process to classify the collected particles as being of one of a set of particle types identifiable by said stored-program.

35. The apparatus of claim 33, further comprising a step for acquiring an image the collected particles and conveying that image to a viewing device.
